(19) United States
(12) Patent Application Publication
(10) Pub. No.: US 2017/0143894 A1
(43) Pub. Date: May 25, 2017

(54) INLINE PATENCY CHECK DEVICE

(71) Applicant: BAYER HEALTHCARE LLC, WHIPPANY, NJ (US)

(72) Inventors: ARTHUR E. UBER, III, PITTSBURGH, PA (US); JOSEPH C. MATOR, PITTSBURGH, PA (US); EDWARD P. LISCIO, MURRSYVILLE, PA (US); FREDERICK W. TROMBLEY, III, GIBSONIA, PA (US); STEPHEN R. SCHULTE, GIBSONIA, PA (US); JOHN D. MARTINO, MORNING VIEW, KY (US)

(21) Appl. No.: 15/319,140

(22) PCT Filed: Jun. 25, 2015
(86) PCT No.: PCT/US15/37668

§ 371 Date: Dec. 15, 2016

Related U.S. Application Data
(60) Provisional application No. 62/018,119, filed on Jun. 27, 2014.

(51) Int. Cl. A61M 5/00; A61M 5/158; A61M 5/145

(52) U.S. Cl. CPC A61M 5/007; A61M 5/14546; A61M 5/158; A61M 2005/1588; A61M 2205/3337; A61M 2005/1402

(57) ABSTRACT

A fluid path set for use with an injection device has a single-patient disposable set and a patency check device. A proximal end of the single-patient disposable set is configured for connecting with the injection device. The patency check device has a proximal end configured for connecting with a distal end of the single-patient disposable set and a distal end configured for connecting with a patient fluid delivery device. The patency check device has a body extending between the proximal end and the distal end. The body has a reversibly expandable interior chamber configured for drawing fluid through the patient fluid delivery device during a patency check. The patency check device further has a locking mechanism configured for controlling the volume of the interior chamber during an injection procedure.

### CROSS-REFERENCE TO RELATED APPLICATIONS

The present application claims priority to U.S. Provisional Patent Application No. 62/018,119, filed on Jun. 27, 2014 and entitled “Inline Patency Check Device”, the disclosure of which is incorporated herein in its entirety.

### BACKGROUND OF THE DISCLOSURE

Field of the Disclosure

The present disclosure is directed to medical fluid delivery applications and, particularly, to an inline patency check device configured for use with a fluid path set of a fluid injection device.

Description of Related Art

In many medical diagnostic and therapeutic procedures, a medical practitioner, such as a physician, injects a patient with a medical fluid. In recent years, a number of injector-actuated syringes and powered injectors for pressurized injection of fluids, such as contrast solution (often referred to simply as “contrast”), have been developed for use in procedures such as angiography, computed tomography (CT), ultrasound, molecular imaging, and magnetic resonance imaging (MRI). In general, these powered injectors are designed to deliver a preset amount of contrast and volumes of saline at preset and controlled flow rates.

In some injection procedures, the medical practitioner places a catheter or a needle connected to tubing, or other fluid delivery connection into a vein or artery of the patient. In some imaging procedures, the contrast or drug is injected through a short catheter in a peripheral vein, for example in the arm or leg, or sometimes through a peripherally inserted central catheter (“PICC”) line or other central catheter. The catheter or the tubing is connected to either a manual or to an automatic fluid injection mechanism. Automatic fluid injection mechanisms typically include at least one syringe connected to at least one fluid injector having, for example, at least one powered linear piston. The at least one syringe includes, for example, a source of contrast and/or a source of flushing fluid. The medical practitioner enters settings into an electronic control system of the fluid injector for a fixed volume of contrast and/or saline and a fixed rate of injection for each.

The injected contrast and/or saline are delivered to a patient's vasculature through the catheter or needle inserted into the patient's body, such as the patient's arm or groin area. A dose of contrast is referred to as a bolus. Once the bolus of contrast is delivered to the desired site, that area is imaged using a conventional imaging technique, such as angiography imaging or scanning, computed tomography (CT), ultrasound, magnetic resonance imaging (MRI), positron emission tomography (PET), and other molecular imaging procedures. The presence of the contrast becomes clearly visible against the background of the surrounding tissue.

Special efforts are often made to assure a proper placement of the catheter or needle in the patient's vasculature. In some cases, extravasation may occur when contrast and/or saline is injected into the surrounding tissue instead of the patient's vasculature. In order to avoid extravasation of contrast and/or saline into the patient's tissue and to assure that the catheter or needle end is not blocked and in fluid communication with the blood vessel, a patency check may be performed. The patency check provides confirmation that the catheter or needle is fluidly connected to the patient's artery or vein. In a typical injection procedure, a patency check may involve the following steps: (a) inserting a catheter or needle into a vein of a patient, (b) connecting a patency check device into the fluid path to the vein of the patient, (c) optionally purging the connection, (d) drawing blood to check for patency, (e) backwashing blood into the bloodstream of the patient with saline, and (f) removing the patency check device from the fluid path. In addition to confirming location in the vessel, the patency check is used to confirm free flow into and out of the catheter. If the catheter or needle has been in the patient for a while, there is risk of a clot forming over the catheter end which may impede flow. If the injection is to be at a relatively high flow rate, for example several ml/s, the operator will generally try to deliver the saline at a high flow rate and may often try to feel or palpate the flow of the liquid in and through the vein to make sure that the vessel can accommodate the flow rates that will be used. After the patency check is complete, the injection procedure may be carried out by ensuring that no significant air will be trapped in the connection, connecting the catheter or needle to the automatic fluid injection mechanism, and initiating the injection procedure.

The existing patency check procedure requires multiple time-consuming steps, such as changing connections between the patency check device and the automatic fluid injection mechanism, that reduce patient throughput and includes several steps when the operator and the environment can be exposed to the patient's blood and the patient's circulatory system can be exposed to pathogens from the environment. For example, for each new patient, a fluid connection must be made with the patency syringe first, before connecting the patient catheter to the automatic fluid injection mechanism. With each new connection, purging must be done to eliminate air from the fluid line, which increases the possibility for operator error.

While conventional patency check devices and methods are known in the medical field, improved patency check devices and methods continue to be in demand.

### SUMMARY OF DISCLOSURE

In view of the disadvantages of the existing patency check procedures and devices, there is a need in the art for improved patency check devices and methods that overcome the deficiencies of the prior art. There is an additional need for improved patency check devices and methods that increase patient throughput while reducing sources of operator error, reducing the risk of exposure of healthcare workers to patient blood, and reducing the risk of exposure of the patient to external pathogens.

In accordance with some aspects of the present disclosure, a fluid path set for use with an injection device may include a single-patient disposable set and a patency check device. The single-patient disposable set may have a proximal end and a distal end. The proximal end of the single-patient disposable set may be configured for connecting with the injection device. A proximal end of the patency check device may be configured for connecting with the distal end of the single-patient disposable set. A distal end of the patency check device may be configured for connecting with a patient fluid delivery device. The patency check device may have a body extending between the proximal end and the distal end of the patency check device. The body may have a reversibly expandable interior chamber configured for drawing fluid through the patient delivery device during a patency check. In certain embodiments, the reversibly expandable interior chamber may include a plunger slidably received within the body between the proximal end and the distal end of the patency check device. The body may further include a locking mechanism configured for controlling the volume of the interior chamber during an injection procedure, such as by locking or otherwise limiting the movement of the plunger relative to the body to prevent unrestrained movement of the plunger during an injection procedure.

In accordance with other aspects of the present disclosure, the locking mechanism may have a first locking member on the plunger and a second locking member on the body of the patency check device. In certain embodiments, the plunger may have a stop that prevents it from coming out of the body when the fluid inside is under pressure, such as a flange, ledge, or stop at the proximal end of the patency check device that projects radially inward into the interior chamber, thereby preventing further proximal movement of the plunger. The proximal end of the patency check device may have a first connector for releasably connecting with the single-patient disposable set or the proximal end of the patency check device may be non-releasably connected to the single-patient disposable set. The distal end of the patency check device may have a second connector for releasably connecting with the patient fluid delivery device or the distal end of the patency check device may be non-releasably connected to the patient fluid delivery device. The plunger may have an at least partially hollow body with a fluid path extending through the at least partially hollow body. An annular seal may be provided between an outer portion of the plunger and an inner wall of the interior chamber of the patency check device. A one-way check valve may be provided within the annular seal, for example to allow fluid to flow from the injector device into the interior chamber of the patency check device. A removable cap may be provided at the distal end of the patency check device. The cap may be porous.

In accordance with other aspects of the present disclosure, a patency check device may include a proximal end configured for connecting with an injection device, a distal end configured for connecting with a patient fluid delivery device, and a body extending between the proximal end and the distal end. The body may have a reversibly expandable interior chamber configured for drawing fluid through a patient fluid delivery device during a patency check. In certain embodiments, the reversibly expandable interior chamber may include a plunger slidably received within the body between the proximal end and the distal end. A locking mechanism may be configured for controlling the volume of the interior chamber during an injection procedure, such as by locking or otherwise limiting the movement of the plunger relative to the body to prevent unrestrained movement of the plunger during an injection procedure. The locking mechanism may have a first locking member on the plunger and a second locking member on the body of the patency check device. In certain embodiments, the plunger may have a stop that prevents it from coming out of the body when the fluid inside is under pressure, such as a flange, ledge, or stop at the proximal end of the patency check device that projects radially inward into the interior chamber, thereby preventing further proximal movement of the plunger. The proximal end of the patency check device may have a first connector for releasably connecting with the injection device or the proximal end of the patency check device may be non-releasably connected to the single-patient disposable set. The distal end of the patency check device may have a second connector for releasably connecting with the patient fluid delivery device or the distal end of the patency check device may be non-releasably connected to the patient fluid delivery device. The plunger may have an at least partially hollow body with a fluid path extending through the at least partially hollow body. An annular seal may be provided between an outer portion of the plunger and an inner wall of the interior chamber of the patency check device.

In accordance with other aspects of the present disclosure, a method of determining patency of an injection site may include inserting a catheter or needle into a blood vessel of a patient, connecting a patency check device into a fluid path between the catheter or needle and an injection device, withdrawing a plunger toward a proximal end of a body of the patency check device to check for patency, wherein patency is verified by drawing blood from the blood vessel into the interior chamber or into the patient fluid path; if patency id present, backwashing blood from the interior chamber or the patient fluid path into the blood vessel of the patient with fluid from the injection device; and locking or otherwise limiting the movement of the plunger relative to the body of the patency check device to prevent unrestrained movement of the plunger during an injection procedure. The patency check device may have a proximal end configured for connecting with the injection device, a distal end configured for connecting with the catheter or needle or a patient fluid path connected to the catheter or needle, and a body extending between the proximal end and the distal end. The body may have an interior chamber configured for drawing fluid from the flood vessel into the patient fluid path or interior chamber during a patency check, a plunger slidably received within the body between the proximal end and the distal end, and a locking mechanism configured for locking or otherwise limiting the movement of the plunger relative to the body to prevent movement of the plunger during an injection procedure. The method may further include priming the fluid path with fluid from the injection device prior to withdrawing the plunger toward the proximal end of the body of the patency check device to check for patency. The method may further include injecting fluid from the injection device after locking the plunger relative to the body of the patency check device.

These and other features and characteristics of the inline patency check device, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only. As used in the specification and the claims, the singular form of “a”, “an”, and “the” include plural referents unless the context clearly dictates otherwise.

### BRIEF DESCRIPTION OF THE DRAWINGS

FIG. 1 is a perspective view of a fluid delivery system according to one aspect.

FIG. 2 is a perspective view of a fluid delivery system according to another aspect.

FIG. 3 is a perspective view of a fluid path set and a single-patient disposable set for use in a fluid delivery system in accordance with one aspect.

FIG. 4A is a partial cross-sectional view of a single-patient disposable set and a patency check device in accordance with a first aspect.

FIG. 4B is a detailed cross-sectional view of the single-patient disposable set and the patency check device shown in FIG. 4A.

FIG. 4C is an exploded partial cross-sectional view of the single-patient disposable set and the patency check device shown in FIG. 4B.

FIG. 4D is a partial cross-sectional view of the patency check device shown in FIG. 4C.

FIG. 4E is an exploded partial cross-sectional view of the patency check device of FIG. 4D.

FIG. 5A is a partial cross-sectional view of a single-patient disposable set and a patency check device in accordance with another aspect.

FIG. 5B is a partial cross-sectional view of a single-patient disposable set and a patency check device in accordance with another aspect.

FIG. 6 is a partial cross-sectional view of a patency check device in accordance with another aspect.

FIG. 7 is a partial cross-sectional view of a patency check device in accordance with another aspect.

FIG. 8 is a partial cross-sectional view of a patency check device in accordance with another aspect.

FIGS. 9A-9D illustrate a patency check procedure in accordance with one aspect.

FIGS. 10A-10D illustrate a patency check procedure in accordance with another aspect.

FIG. 11 is an exploded perspective view of a patency check device in accordance with another aspect.

FIGS. 12A-12F are cross-sectional perspective views of a patency check procedure using the patency check device shown in FIG. 11.

### DETAILED DESCRIPTION

For purposes of the description hereinafter, the terms “upper”, “lower”, “right”, “left”, “vertical”, “horizontal”, “top”, “bottom”, “lateral”, “longitudinal”, and derivatives thereof shall relate to the disclosure as it is oriented in the drawing figures. When used in relation to an inline patency check device, the term “proximal” refers to a portion of an inline patency check device nearest to a user, such as a medical technician. The term “distal” refers to a portion of an inline patency check device farthest away from a user. The term “radial” refers to a direction in a cross-sectional plane normal to a longitudinal axis of an inline patency check device extending between proximal and distal ends. The term “circumferential” refers to a direction around an inner or outer surface of a sidewall of an inline patency check device. The term “axial” refers to a direction along a longitudinal axis of an inline patency check device extending between the proximal and distal ends. It is to be understood, however, that the disclosure may assume alternative variations and step sequences, except where expressly specified to the contrary. It is also to be understood that the specific devices and processes illustrated in the attached drawings, and described in the following specification, are simply exemplary aspects of the disclosure. Hence, specific dimensions and other physical characteristics related to the aspects (i.e., aspects, variants, variations) disclosed herein are not to be considered as limiting.

Referring to the drawings in which like reference characters refer to like parts throughout the several views thereof, the present disclosure is generally directed to an inline patency check device configured for use with a fluid path set of a fluid injection device.

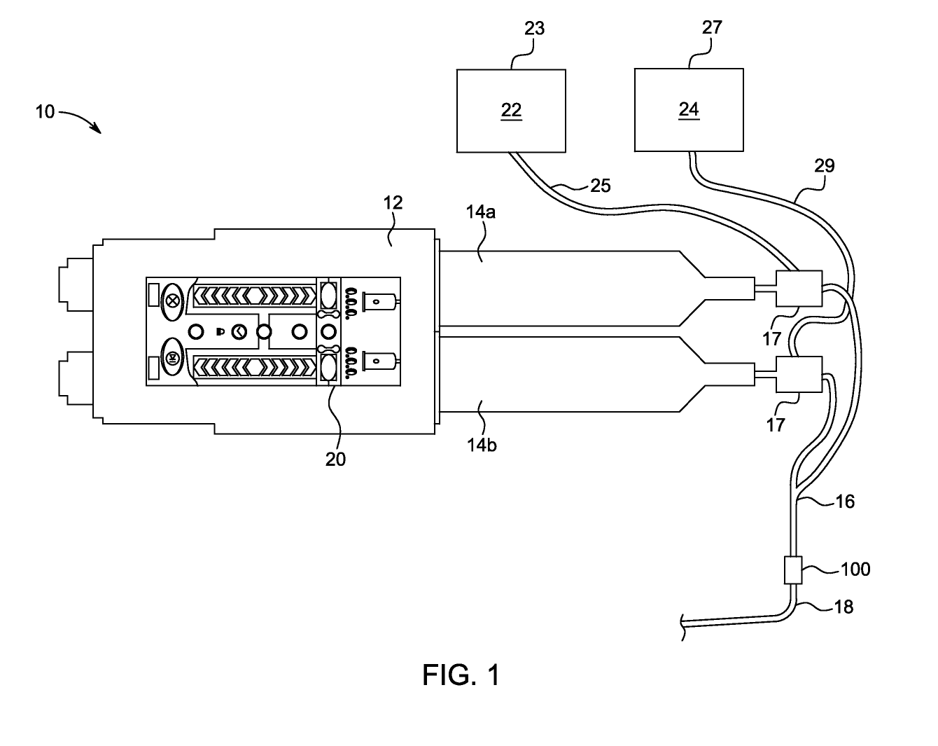

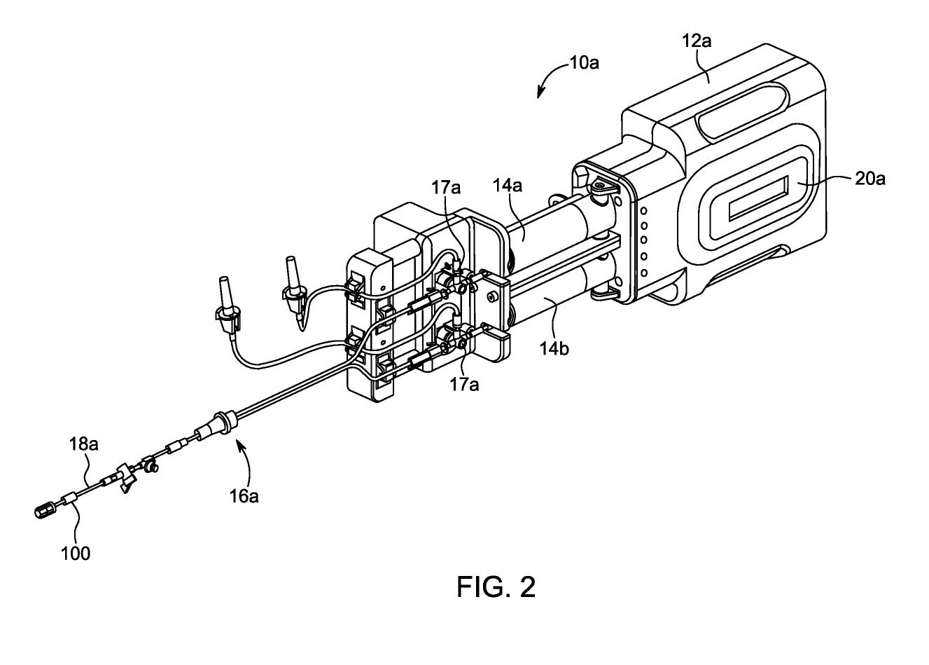

FIGS. 1-2 are perspective views of a fluid delivery system 10 having an inline patency check device 100 (hereinafter referred to as “patency check device 100”) according to one aspect. The fluid delivery system 10 is adapted for delivering at least one fluid to a patient during a medical injection procedure. For example, the fluid delivery system 10 may be used during an angiographic, CT, MRI, or other medical imaging procedure to inject a contrast solution and/or a flushing agent, such as saline, into the body of a patient. In other embodiment, the patency check device 100 may be used to ensure patency during fluid delivery of any medical fluid to a patient. An example of such a fluid injection or delivery system is disclosed in U.S. Pat. No. 7,094,216, and assigned to the assignee of the present application, the disclosure of which is incorporated herein by reference in its entirety. Additional examples of fluid delivery systems are disclosed in the following references: U.S. Pat. No. 7,556,619; U.S. Pat. No. 8,337,456; U.S. Pat. No. 8,147,464; and U.S. Pat. No. 8,540,698, each of which are assigned to the assignee of the present application and the disclosures of which are incorporated herein by reference in their entireties. The patency check device 100 is generally adapted to interface with one or more components of the fluid delivery system 10 to aid in confirming that fluid connection with the patient's vascular system is patent.

The fluid delivery system 10 generally includes a powered fluid injector 12 that is adapted to support and actuate at least a first syringe 14a configured for receiving and delivering a first injection fluid 22 stored in a first fluid container 23 via a first delivery line 25 for injection to a patient during a medical imaging procedure. The fluid delivery system 10 may further include at least a second syringe 14b configured for receiving and delivering a second injection fluid 24 stored in a second fluid container 27 via a second delivery line 29. The second injection fluid 24 may be mixed with the first injection fluid 22 or delivered separately to the patient. The injector 12 is generally used to supply at least the first and second injection fluids 22, 24 under pressure to a fluid path set 16 and, ultimately, the patient. Other embodiments of the fluid injector 12 may include at least a third syringe (not shown). According to other embodiments, the injector 12 may include at least one pump and in certain aspects a plurality of pumps, such as piston pumps and/or peristaltic pumps. For example, in one aspect the injector 12 may be at least a dual-syringe injector, wherein at least two fluid delivery syringes are oriented in a side-by-side or other spatial relationship and which are separately actuated by respective piston elements associated with the injector 12. In another aspect, the injector 12 may be a single- or dual-pump injector, wherein one or two pumps, such as piston pumps and/or peristaltic pumps, are separately actuated and controlled to deliver the first fluid 22 and the second fluid 24 to the patient. In certain embodiments of a single-pump injector, the second fluid may be delivered by a syringe. In one aspect, the second injection fluid 24 may be delivered by way of a pump, such as a piston pump or a peristaltic pump. In another aspect, the second injection fluid 24 may be delivered by way of a second powered injector, as described in greater detail below. The injector 12 may be controlled by a hand controller to supply the first and second injection fluids 22, 24 at discrete and preselected flow rates. In other embodiments, the injector 12 may be controlled by a processor, such as a fluid control module, having programmed and/or operator entered instructions for supplying the first and second injection fluids 22, 24 at discrete and preselected flow rates.

The injector 12 may be operatively associated with a fluid control module 20. The fluid control module 20 may be adapted for controlling the operation of the fluid delivery system 10 by allowing the user to manually select the injection parameters, or select a pre-defined injection protocol. In some aspects, the fluid control module may have one or more buttons, knobs, touch pads, displays, switches, dials, or other input and/or output devices to allow the user to user to manually select the injection parameters, or select a pre-defined injection protocol. Alternatively, this functionality may reside with an external control unit or with the powered injector 12. In either case, the fluid control module 20 controls, for example but not limited to, the injection pressure, the volumes of the various fluids to be delivered to the patient, for example, of the first and/or second injection fluids 22, 24 to be delivered to the patient, and the ratio of the various fluids to be delivered, such as the first injection fluid 22 relative to the second injection fluid 24.

The fluid delivery system 10 is generally adapted to connect to a fluid path set 16 for delivering the first and second injection fluids 22, 24. The flow of the first injection fluid 22 from the first syringe 14a and the second injection fluid 24 from the second syringe 14b is regulated by the fluid control module 20, which controls the various pistons, plungers, drives, actuators, pumps, valves, and/or flow regulating structures to regulate the delivery of first and second injection fluids 22, 24 to the patient based on user selected injection parameters, such as total injection volume, rate of injection, timing of injection, injection pressure, and/or ratio of the first injection fluid 22 and second injection fluid 24. The fluid path set 16 may further connect the at least one syringe 14a, 14b to a single-patient disposable set (SPDS) 18 via a connector 17. The SPDS 18 may be connected to the patient for supplying the first injection fluid 22 and second injection fluid 24 to the patient. The patency check device 100 may be disposed within the fluid path, for example in the fluid path set 16 or, for embodiments including an SPDS, in-line within or distal to the SPDS 18.

The first and second syringes 14a, 14b may have the same or a different size relative to each other. For example, in certain aspects, one of the first syringe 14a and the second syringe 14b may be larger relative to the other of the first syringe 14a and the second syringe 14b to receive a larger volume of fluid therein. A suitable multi-syringe fluid injector for use with the above-described system is described in U.S. Patent Application Publication No. 2012/0123257, filed on Jan. 24, 2012, and is assigned to the assignee of the present application, the disclosure of which is incorporated herein by reference in its entirety. Other relevant multi-fluid delivery systems are found in U.S. Patent Application Publication No. US 2004/0064041, filed on May 30, 2002, U.S. Patent Application Publication No. 2005/0113754, filed Nov. 25, 2003, and International Patent Application Publication No. WO 2012/155035, filed on May 11, 2012, all of which are assigned to the assignee of the present application, and the disclosures of which are incorporated herein by reference.

In yet another aspect, a three-fluid delivery system (not shown) may be provided. Similar to power-operated fluid delivery systems described with reference to FIGS. 1-2, a three-fluid delivery system may include a first injector or pump adapted to deliver a first injection fluid 22 (shown in FIG. 1), such as a contrast medium, a second injector or pump adapted to deliver a second injection fluid 24 (shown in FIG. 1), such as saline, and a third injector or pump adapted to deliver a third injection fluid, such as, for example a second contrast medium or a contrast medium of the first injection fluid having a different concentration. Alternatively, single or dual injectors may be attached to two or more fluid sources, for example by one or more fluid paths. A fluid path set is provided for delivering and mixing the first, second, and/or third injection fluids in a desired ratio prior to being delivered to a patient. The fluid path set may comprise a patency check device 100 as described herein. An exemplary three-fluid delivery system is disclosed in FIGS. 60-62 of U.S. Patent Application Publication No. 2012/0123257 discussed herein.

In another aspect, a manually-controlled fluid delivery system (not shown) may be provided. Similar to power-operated fluid delivery systems described with reference to FIGS. 1-2, a manually-controlled fluid delivery system may include a first injector adapted to actuate a first syringe storing a first injection fluid 22 (shown in FIG. 1), such as a contrast medium, for injection to a patient during a medical procedure. The manually-controlled fluid delivery system may also include a second injector adapted to actuate a second syringe storing a second injection fluid 24 (shown in FIG. 1), such as saline. A fluid path set is provided for delivering and mixing the first injection fluid 22 and the second injection fluid 24 in a desired ratio prior to being delivered to a patient. The fluid path set may comprise a patency check device 100 as described herein. An exemplary manually-controlled fluid delivery system is disclosed in U.S. Patent Application Publication No. 2014/0107480, filed Jan. 31, 2013, assigned to the assignee of the present application, the disclosure of which is incorporated herein by reference.

The following operational discussion of the patency check device 100 will be with exemplary reference to an imaging procedure involving the fluid delivery system 10 and how the patency check device 100 contributes to checking the patency of the fluid connection between the fluid delivery system 10, 10a and the patient's vasculature. Other injection procedures including those with other medical fluids may utilize the patency check device 100 describe herein, in a similar manner. In typical imaging procedures, the first injection fluid 22 is a contrast solution and the second injection fluid 24 or flushing agent is saline. The contrast solution typically has higher viscosity and specific gravity compared to saline. Depending on the medical procedure, various other medical fluids can be used as the first injection fluid 22 and the second injection fluid 24.

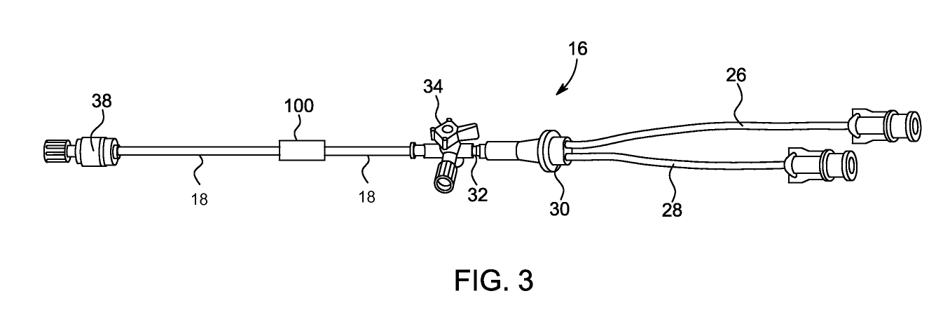

With reference to FIG. 3, the fluid path set 16 is shown separate from the fluid delivery system 10. The fluid path set 16 includes a first fluid line 26 in fluid communication at its proximal end with the source of the first injection fluid 22 and a second fluid line 28 in fluid communication at its proximal end with the source of the second injection fluid 24. The first and second fluid lines 26, 28 act as fluid conduits for delivering the first and second injection fluids 22, 24, respectively, from the source of each respective fluid. Distal ends of each of the first and second fluid lines 26, 28 are in fluid communication with a fluid connector 30 that combines the first and second fluid lines 26, 28 into a single fluid outlet line 32. One or more valves 34, such as a stopcock, may optionally be provided within the fluid path set 16 to selectively block the passage of the first and/or second injection fluid 22, 24 through the fluid path set 16. For example, a one-way check valve 34 may be provided on the fluid outlet inline 32 to prevent the contrast and/or saline from flowing back into the fluid connector 30 and into first and second fluid lines 26, 28. According to certain embodiments, single fluid outlet line 32 may be a single patient disposable set (SPDS), whereas fluid lines 26, 28, may be part of a multi-patient disposable set (MPDS). While various embodiments of the patency check device and fluid path sets described herein are disclosed with reference to a contrast or imaging agent administration procedure, the patency check devices and fluid path sets may be used with any appropriate medical injection procedure where patency between the patients vasculature and the injection fluid is desired or required and the present description is intended to capture such uses.

The distal end of the fluid connector 30 may be releasably connected to a SPDS 18 (shown in FIG. 1) to deliver the mixed solution of the first and second injection fluid 22, 24 to the patient. The connector 30 may be a two-part connector having a first component provided on the distal end of the MPDS and a corresponding second component provided on a proximal end of the SPDS 18 (shown in FIG. 3). In some aspects, the connector 30 may be bonded to the distal end of the MPDS and the proximal end of the SPDS 18 and fluid path 32 by a conventional UV bonding technique. Alternatively, the connector 30 may be coupled to the distal end of the MPDS and the proximal end of the SPDS 18 and fluid path 32 by an over-molding technique. In some aspects, the connector 30 may have a luer-type connection at the distal end of the MPDS configured for coupling with a corresponding luer-type connection on the SPDS 18 and fluid outlet line 32. One of ordinary skill in the art will appreciate that the distal end of fluid outlet line 32 and the proximal end of the SPDS 18 may be removably coupled in a number of art-recognized ways. According to various aspects, the patency check device 100 may be located in fluid path set 16, alternatively may be located in SPDS 18, or at the connection between fluid path set 16 and SPDS 18, or between SPDS 18 and the patient catheter.

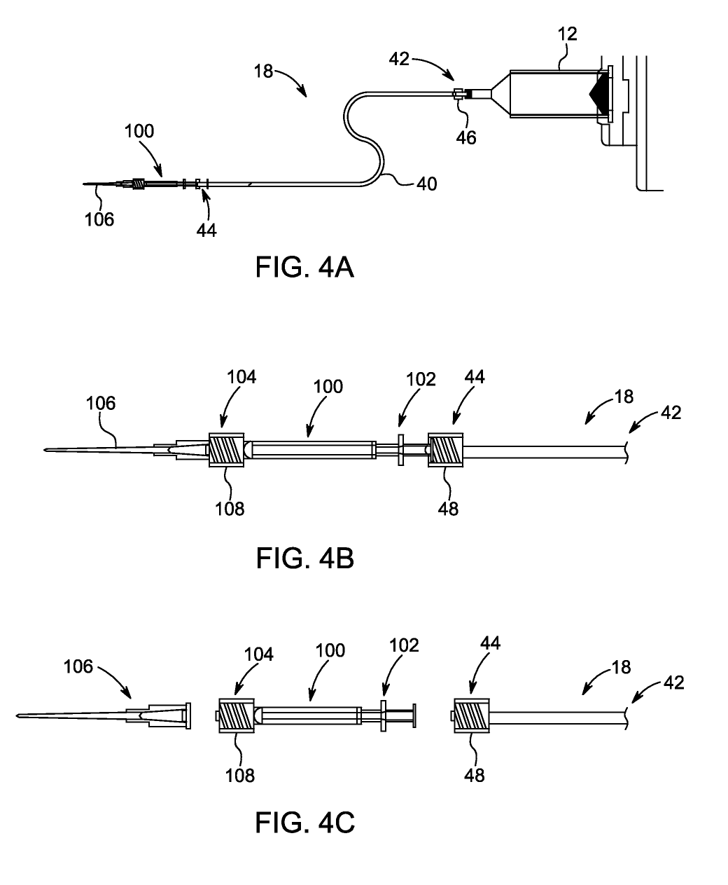

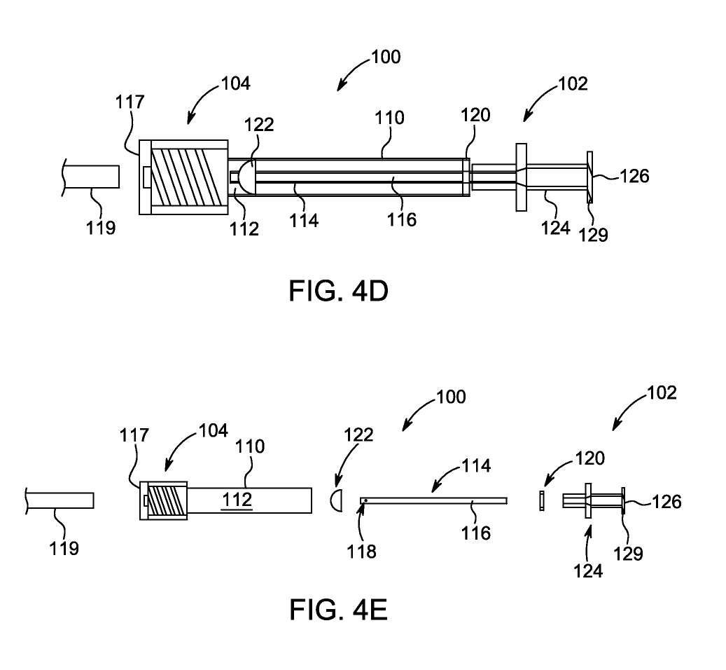

With reference to FIG. 4A, according to one aspect, the SPDS 18 has an elongated body 40 having a proximal end 42 configured for connection with the distal end of the fluid outlet line 32, as shown in FIG. 1, or to a distal end of the injector 12, as shown in FIG. 4A. The SPDS 18 further has a distal end 44 opposite the proximal end 42. The elongated body 40 has a substantially tubular cross-section with a central lumen extending therethrough. The central lumen of the SPDS 18 is configured for delivering fluid between the proximal end 42 and the distal end 44. The proximal end 42 has a connector 46 configured for releasably coupling with the connector 30 provided on the distal end of the fluid outlet line 32 (FIG. 3), or for releasably coupling with the injector 12. In some aspects, the distal end 44 of the SPDS 18 is configured for being releasably or non-releasably connected with the patency check device 100. In other aspects, the distal end 44 of the SPDS 18 is configured for being releasably or non-releasably connected with a connector, a needle, or additional tubing, as described herein, wherein the patency check device is releasably or non-releasable connected within the fluid path of SPDS 18.

With reference to FIGS. 4B-4C, a portion of the SPDS 18 is shown in combination with an aspect of the patency check device 100 in accordance with certain aspects. The patency check device 100 may be connected to the distal end 44 of the SPDS 18 by way of a releasable connection. For example, the distal end 44 may have a male luer connector 48 that is configured for releasably coupling with a corresponding connection provided on the proximal end of the patency check device 100 (shown in FIG. 4C). In some aspects, the patency check device 100 may be connected to the distal end 44 of the SPDS 18 by way of a non-releasable connection. For example, the SPDS 18 may be manufactured with the patency check device 100 built into the fluid path or may be non-releasably connected to the fluid path by adhesive or welding.

With continuing reference to FIGS. 4B-4C, the patency check device 100 has a proximal end 102 configured for connecting to the distal end 44 of the SPDS 18. The patency check device 100 further has a distal end 104 opposite to the proximal end 102. The distal end 104 of the patency check device 100 may be configured for connecting to a needle 106 having a cannula with a pointed distal end configured for accessing the patient's vasculature or may be connected to a connector on a patient catheter in fluid connection with the patient's vasculature. In various aspects, the distal end 104 of the patency check device 100 may be connected with the needle 106 or catheter connector by way of a releasable or non-releasable connection. In one aspect, the distal end 104 of the patency check device 100 has a luer-type connector 108 configured for releasably coupling with a corresponding luer-type connector on the needle 106. Alternatively, the distal end 104 of the patency check device 100 may be connected with a second fluid tubing path of SPDS 18, wherein the second fluid tubing path is in fluid connection with a needle 106 having a cannula with a pointed distal end configured for accessing the patient's vasculature or may be connected to a connector on a patient catheter in fluid connection with the patient's vasculature.

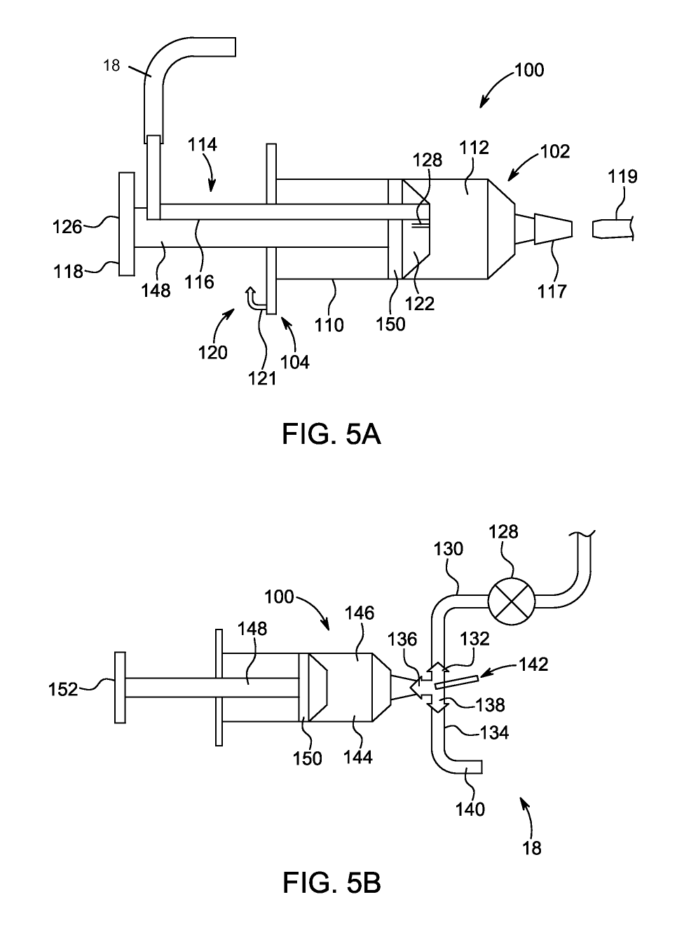

With reference to FIGS. 4D-4E, according to various aspects, the patency check device 100 has a tubular body 110 with a reversibly expandable interior chamber 112 for drawing fluid from a patient's vasculature through the patient fluid delivery device during the patency check. According to certain embodiments, a plunger tube 114 is slidably received within the interior chamber 112 of the tubular body 110. The plunger tube 114 has an at least partially hollow body with a fluid path 116 extending between the proximal and distal ends of the plunger tube 114. Fluid connection between the plunger tube 114 and the SPDS 18 may be made in an axial direction, such as shown in FIGS. 4D-4E. Alternatively, in some aspects, the fluid path 116 may be connected to the SPDS 18 at least partially on a radial side of the patency check device 100 before fluid flow is diverted in an axial direction, as shown in FIG. 5. A cap 117 may be provided at the distal end 104 of the patency check device 100 to prevent contamination of the distal end 104 prior to connection with the patient fluid delivery tubing 119. The cap 117 may be removable prior to connecting the distal end 104 of the patency check device 100 with the patient tubing 119.

With continuing reference to FIGS. 4D-4E, the plunger tube 114 has a first locking member 118 provided on an outer surface thereof. The first locking member 118 is configured for interacting with a second locking member 120 provided at a proximal end 102 of the tubular body 110. In one aspect, the second locking member 120 has an annular structure with a locking feature that corresponds to the first locking member 118 on the plunger tube 114. The second locking member 120 may be permanently coupled to the proximal end 102 of the tubular body 110. Once connected, the connection between the first and second locking members 118, 120 may be non-reversible to prevent unrestrained movement or separation of the two elements (i.e., body 110 and plunger tube 114) of the patency check device 100 when under the pressure of an injection through the patency check device 100, as described herein. In an alternative embodiment, the interaction of the two locking members 118 and 120 may limit the proximal movement the plunger tube 114 but may not permanently lock the plunger tube 114 with respect to the tubular body 110. For example, the second locking member 120 may be a stop, flange or ledge projecting radially into the interior chamber which stops further proximal movement of plunger tube 114, when contact between the second locking member 120 and the plunger tube 114.

With continuing reference to FIGS. 4D-4E, the patency check device 100 further includes an annular seal 122 disposed around the distal end of the plunger tube 114. The annular seal 122 extends around the exterior surface of the plunger tube 114 and slidably contacts the interior surface of the wall of the plunger body 110 to provide a distal seal for the interior chamber 112. The proximal end of the plunger tube 114 has an activation member 124. In some aspects, the activation member 124 has a hollow interior and is configured for connecting at its distal end to the proximal end of the plunger tube 114 in line with the fluid path 116 (shown in FIG. 1). In this aspect, the user may grip the activation member 124 and the plunger body 110 to control the volume of interior chamber plunger tube 112 during a patency check, for example to pull them apart axially to expand the volume within interior chamber 112 or push them together axially to reduce the volume within interior chamber 112 in the patency check device 100. Axial expansion of interior chamber 112 will draw blood from the patient's vasculature system into the patient fluid path 119 or into interior chamber 112 if patency is present. The proximal end of the activation member 124 has a connector 129 that is configured for releasably coupling with the luer connector 48 on the distal end 44 of the SPDS 18 to provide fluid connectivity between the SPDS 18 and the patient catheter or patient fluid path 119 through patency check device 100. In some aspects, the proximal end of the activation member 124 has a connector 129 that is configured for being permanently coupled with the distal end 44 of the SPDS 18. One of ordinary skill in the art will appreciate that the connection between the proximal end of the activation member 124 and the distal end 44 of the SPDS 18 can be made in a number of ways. As described herein, the plunger tube 114 of the patency check device 100 can be moved axially relative to the tubular body 110 to draw fluid into the patient fluid path 119 or the interior chamber 112 (by moving the activation member 124 axially in a proximal direction) or to expel fluid from the interior chamber 112 (by moving the activation member 124 axially in a distal direction).

In some aspects, the patency check device 100 may allow injection of fluid through the device, thereby eliminating the need for preparation, connection, and disconnection of a separate patency check device. For the patency check device 100 to be able to pull blood from the patient or deliver saline to the patient, the proximal end must be connected to a fluid source that is relatively non-compressible or expansive. That is, when patency check device 100 is expanded from its compressed length to its expanded length, most of the fluid comes through the connector 108. If the path to the patient is patent, blood will flow from the patient into the patient fluid path 119 or interior chamber 112 of patency check device 100 through the connector 108. To test for sufficient vessel capacity to carry fluid away from the injection site, a short injection of saline can be made using the injector. Alternatively, the patency check device 100 can be quickly compressed to deliver some of the fluid from the interior chamber 112 into the patient. When this bolus injection is made, the interior chamber 112 of the patency check device 100 may be expanded again, forcing the plunger tube 114 and first locking member 118 until first locking member 118 is engaged by second locking member 120 as described herein. Once the patency check device 100 is in a locked configuration or further proximal movement of plunger tube 114 is prevented, fluid from the injector may be delivered through the patency check device 100 to the patient. In some aspects, the SPDS 18 may include one or more one-way check valves to prevent flow of blood and other potential pathogens from the patient to any part of the multi-patient fluid path set 16 that may be used with additional patients. In some aspects, a check valve may be provided proximal to the patency check device 100 so that the expanding of the patency check device 100 will pull blood from the patient's vessel and not from the injection device. If check valves are provided distally of the patency check device 100, they may be bypassable check valves as described in U.S. Application Publication No. 2008/0172006, the disclosure of which is incorporated by this reference.

FIG. 5A shows an alternative aspect of fluid flow through the patency check device 100 which incorporates a one-way check valve 128 into the plunger sealing member 122. The plunger tube 114 carries the fluid, such as saline and/or contrast, into the patency check device 100. In this aspect, the fluid path 116 extends through the plunger sealing member 122. The plunger sealing member 122 contains one or more slits, duckbill valves, leaves, balls, or other sealing elements that cooperate with support member 148 to allow one-way flow from fluid path 116 in a direction toward the patient and prevent flow from the patient into fluid path 116 into interior chamber 112. The patency check device 100 incorporating the one-way check valve 128 has a significant cost savings over a separate check valve within the fluid path as it only has three parts (not including the optional cap 117). In addition, it prevents contamination of more proximal fluid path elements while providing for a patency check.

In operation, the patency check device 100 of FIG. 5A is similar to that of FIG. 4, except that the first and second locking members 118 and 120, respectively, are not locked together until the plunger tube 114 is pushed far enough forward for the second locking member 120 to latch onto first locking member 118. Once engaged, the locking members may be configured so the latching may be irreversible and the device 100 stays in the compressed position, or alternatively, the operator may be able to disengage the locking mechanism, if desired. According to various aspects, the locking mechanism may be configured so the patency check device 100 will not expand (i.e., plunger tube 114 will not be forced in a proximal direction relative to the body 110) under the pressure of a normal injection procedure and contrast and/or flushing fluid delivered into the patency check device 100 from the injector will exit to the patient through patient tubing 119. With reference to FIG. 5B, a patency check device other than an “in-line” or “flow-through” device as described herein, is shown. The patency check device may have an external 3-way valve 138 having a distal tube 130 in fluid connection with a check valve 128 for connecting to a patient, a proximal tube 140 for connecting to a fluid injector, and a port 136 for connecting to the patency check device 100. The 3-way valve 138 may have a selector switch 142 to selectively block the proximal tube 140 during a patency check and the port 136 during an injection procedure. The device of FIG. 5B differs from those of the present disclosure at least in that it is not in line with the fluid path allowing fluid to flow through the patency check device.

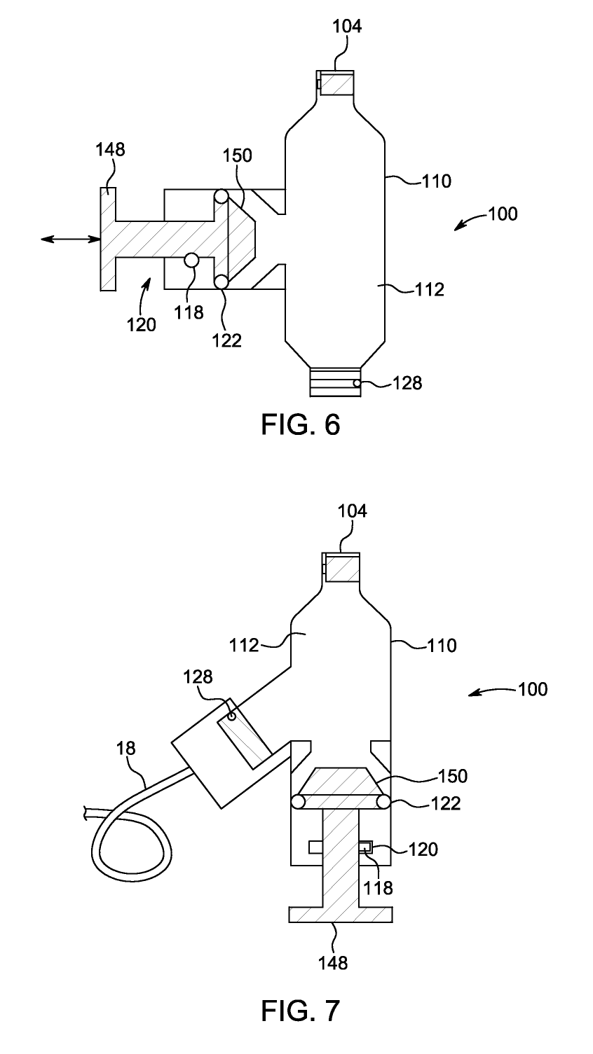

With reference to FIGS. 6-8, various aspects of the patency check device 100 are illustrated. In each of the aspects shown in FIGS. 6-8, the patency check device 100 is configured for connection between the distal end 44 of the SPDS 18 (not shown in FIGS. 6-8) and the proximal end of the patient fluid path 119 or patient catheter or the needle 106 (not shown in FIGS. 7-9). In the aspect shown in FIG. 6, the patency check device 100 includes a tubular body 110 with an interior chamber 112. A plunger rod 148 having a plunger 150 is provided on a lateral side of the tubular body 110. A seal 122 seals between the plunger 150 and the tubular body 110. The plunger rod 148 is configured to draw fluid into or expel fluid from the interior chamber 112 during the patency check procedure. Once the patency check procedure has been completed, the plunger rod 148 is locked by way of a locking mechanism, for example by engaging first locking member 118 with second locking member 120 to prevent its movement during a fluid injection procedure.

With reference to FIG. 7, the arrangement of the fluid path and the plunger rod 148 has been inverted relative to the aspect shown in FIG. 6 but operates in a similar manner as the patency check device 100 described with reference to FIG. 6. According to certain aspects illustrated in FIGS. 6-7, the patency check device 100 may further comprise a check valve 128, for example at the connection between the patency check device 100 and SPDS tubing set 18 or MPDS to prevent fluid, such as blood flowing from the interior chamber 112 into the SPDS tubing set 18 or into the MPDS.

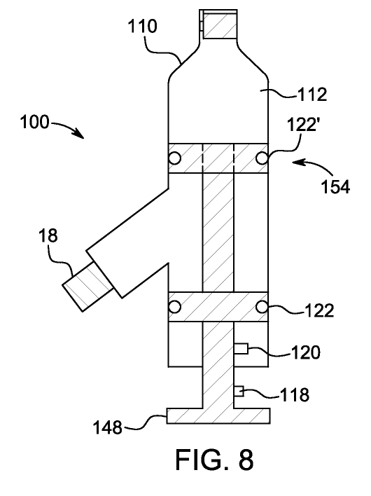

FIG. 8 illustrates an aspect of the patency check device 100 having a check valve 154 within the interior chamber 112 of the tubular body 110. Seal 122 prevents the flow of fluid around the check valve 154. A second seal 122′ at the proximal end of the tubular body 110 allows the plunger rod 148 to move within and exit the tubular body 110 without leakage of fluid. In this aspect, the check valve 154 can be any check valve known to one skilled in the art, for example ball valve, duckbill valve, umbrella valve, or disc check valve. According to one aspect, a single elastomeric element 122 may form both seals 122 and 122′ and the elastomeric element of the check valve 154. In a further aspect, not shown, the two seals 122 and 122′ may be formed by the same elastomeric element and the check valve 154 may be a separate element or elements or may be formed integral therewith. According to other aspects, the patency check device 100 may be located within the SPDS tubing set 18 or between multi-patient tubing set 16 and SPDS tubing set 18.

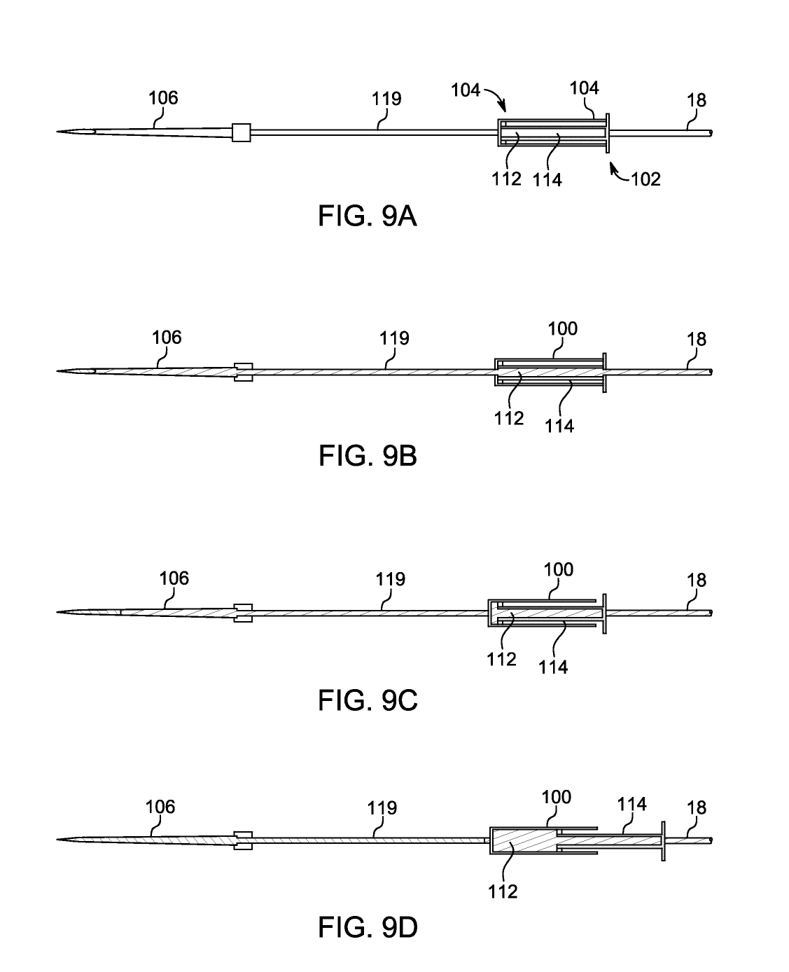

With reference to FIGS. 9A-9D, a patency check procedure using an aspect of the patency check device 100 will be described in accordance with one aspect. As shown in FIG. 9A, the patency check device 100 is connected at its proximal end 102 to the SPDS 18. The distal end 104 of the patency check device 100 is connected to the patient tubing 119 which is connected at its distal end to the catheter or needle 106. In some aspects, the distal end 104 of the patency check device 100 is connected directly to the catheter or needle 106. The assembly is first purged of air and primed with saline (see FIG. 9B). The needle 106 is inserted into the patient's vein or artery (FIG. 9C) and the plunger tube 114 (or plunger rod 148) is drawn in a proximal direction to draw fluid, i.e., blood, into the interior chamber 112 of the patency check device 100 (FIG. 9D). In some aspects, fluid is drawn only in the patient tubing 119, which may be transparent or translucent so that the presence of blood may be observed during patency testing. Once patency is confirmed by visually seeing blood within the patient tubing and/or the interior chamber 112, the plunger tube 114 (or the plunger rod 148) is locked or its movement restrained to prevent its further unrestrained movement during an injection procedure. If the patency check fails, such as when no blood is visualized within the patient tubing 119 and/or the interior chamber 112, the needle 106 may be reinserted into a different location on the patient's body and the patency check repeated until patency is confirmed. After patency is verified, the patency check device 100 may be locked to prevent saline and/or contrast from expanding the patency check device 100 during the injection procedure or ejecting plunger tube 114 from interior chamber 112. In some aspects, the plunger tube 114 (or the plunger rod 148) may be automatically locked once the plunger tube 114 (or the plunger rod 148) is advanced to a predetermined position relative to the body 110 (or the barrel 144). Optionally, the locking of the plunger tube 114 (or the plunger rod 148) prevents the reuse of the patency check device 100, thereby preventing use of the patency check device on a different patient in a second procedure. After the patency check procedure is completed and patency with the patient's vasculature has been verified, an injection procedure may be performed.

While FIGS. 9A-9D illustrate an aspect where the patency check device 100 is initially empty before being primed with saline, in some aspects, patency check device 100 may be prefilled with saline. In such aspects, the patency check device 100 may be filled with saline as the injection system is primed, or the patency check device 100 may be prefilled prior to making the connection with the SPDS 18. As with other aspects described herein, the prefilled patency check device 100 desirably has an auto-locking mechanism that prevents contrast and/or saline from expanding or filling the interior chamber 112 during the injection procedure. The patency check device 100 may be releasably or non-releasably connected with the SPDS 18 and/or the patient tubing 119.

To further expand on the steps for use of the patency check device according to various aspects described herein, a first step may be to connect the patency check device 100 to a source of fluid, such as a priming fluid, for example saline. In certain embodiments, this may be accomplished by attaching SPDS 18 having a patency check device 100 to a MPDS. The tubing set including the SPDS 18, patency check device 100 and MPDS may be primed with saline or other suitable medical fluid to remove air bubbles. For example, a second step may be to push or pull priming fluid into the patency check device 100 and tubing. This can be done by the injector upstream of the device, or in the aspect of FIG. 5, by the device itself because the check valve 128 will pull fluid from upstream and deliver it downstream, provided the fluid source allows fluid to flow from it without creating a significant vacuum. As the fluid flows into the patency check device 100, the air may leave through the open distal end, through a porous cap 117 (shown in FIGS. 4D-4E), or may remain in the patency check device 100. Once fluid has reached and sufficiently filled the patency check device 100, the operator may then manually manipulate the patency check device 100 and remove any remaining air. If there is a cap 117, it can be removed before or after this step. The distal tip may then be connected to the patient's IV fluid path, needle, or previously primed patient line 119. To check for patency, the volume of the interior chamber 112 is expanded and blood can be pulled back from the patient using the patency check device 100 and/or fluid can be pushed into the patient to confirm patency. The patency check device 100 can then optionally be locked into a fixed position as described herein and the injection procedure of contrast and saline can be commenced. After the injection procedure, the SPDS 18 and, the patency check device 100, optionally still connected to the patient's IV fluid path can be removed and properly disposed of.

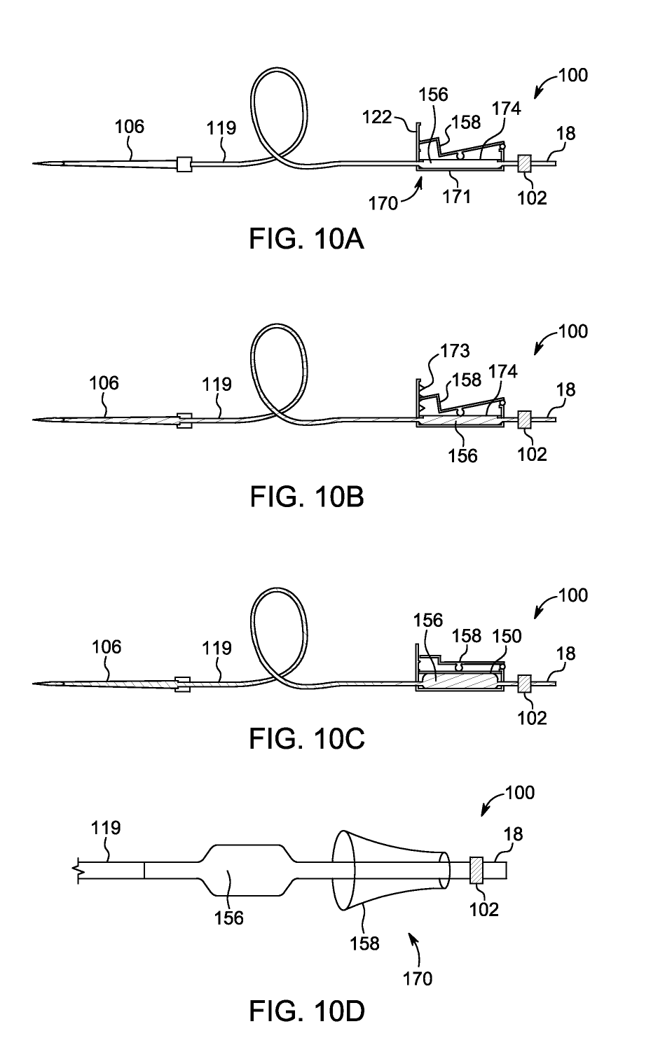

With reference to FIGS. 10A-10C, the patency check device 100 is shown in accordance with a further aspect. With reference to FIG. 10A, in this aspect the patency check device 100 is not syringe-like with a sliding sealable interface. Instead, the internal chamber 112 may be defined by an expandable and pressurizable fluid tube, container, or chamber 156. The fluid chamber 156 may be constrained by housing 170. For example, the constraints may include a base wall 171 and a top wall 174 which are connected to the walls of or are pressed against the chamber 156 by pressure member 158. The housing 170 further has a latch 172 with one or more latching elements or teeth 173 to hold pressure member 158 in one or more fixed positions. The patency check device 100 is at its proximal end 102, connected to or a part of the SPDS 18. In other aspects, the patency check device 100 may be connected to the SPDS 18 at its radial side using the appropriate connector 108 as described herein. In some aspects, the distal end 104 of the patency check device 100 is connected directly to the needle 106 or patient catheter.

As shown in FIG. 10B, the assembly may then be purged of air and primed with saline. Priming removes air and fills the interior chamber 156 and patient tubing 119 with fluid such as saline or other suitable medical fluid. The needle 106 is inserted into the patient's vein or artery and a pressure mechanism 158 is released from latch 172 to expand the volume of the interior chamber 156. The expansion of the interior chamber 156 draws fluid into the interior chamber 156 of the patency check device 100 (FIG. 10C), for example blood from the patient and fluid from the upstream injector if possible. Once patency is confirmed by visually seeing blood within the interior chamber 156 and/or in patient tubing 119, the interior chamber 156 is locked to prevent its further expansion for example against latch 173, or optionally interior chamber 156 can be compressed by applying pressure on pressure mechanism 158 thereby compressing top wall 174 towards base wall 171 to inject the blood and optionally some additional fluid from interior chamber 156 into the patient injection site. If the patency check fails, the needle 106 may be reinserted into a different location on the patient's body and the patency check repeated. After patency is verified, the patency check device 100 may be locked to prevent saline and/or contrast from further filling or rupturing the interior chamber 156 of patency check device 100 during the injection procedure, for example by locking pressure mechanism 158 in the compressed or expanded position, or any position therebetween. After the patency check procedure is completed and patency with the patient's vasculature has been verified, an injection procedure may be performed.

FIG. 10D illustrates an additional aspect of a patency check device 100 in which the interior chamber 156 is an enlarged or ballooned segment in a tube. Interior chamber 156 may be manufactured as an integral part of the tube 119 during extrusion, or may be manufactured as a separate piece and subsequently assembled into a unit. Interior chamber 156 may be compressed by hand and preferably expands due to the resiliency of the wall material. The material properties, wall thickness, and ID of interior chamber 156 can be selected to withstand the expected injection pressure without reinforcement or constraint, or the interior chamber 156 walls can be reinforced with braiding, sheathing, for example with a thin PET balloon, or with a housing 170 arranged concentrically around and slidable on the fluid path. In this aspect, the housing 170 may comprise a single piece of material of sufficient strength which is configured to be slid over the interior chamber 156 to compress or prevent further expansion of at least a portion of interior chamber 156. When slid onto the interior chamber 156, it is held in place, for example by friction against the interior chamber outer wall, or through some other mechanical locking means, such as the ratchets of FIG. 10B. The patency check device 100 of FIG. 10D provides a variety of lockable and stable compressive and expansive states between the two extremes of fully compressed and fully expanded.

In operation, there may be times when the operator permits, desires and/or can cause the patency check device 100 to transition between different volumes to pull in or be filled with priming fluid, to pull in blood from the patient, to expel air or fluid, and/or to deliver fluid to the patient. The various locking, latching, and ratchet mechanisms of various aspects enable the volume or position of the patency check device 100 to be controllably restrained from changing volume of interior chamber 112, 156 when desired by the operator. For example, a patency check device 100 as shown in FIG. 10D may include a sufficiently strong walled interior chamber may have no fixed positions. Alternatively, the interior chamber 156 may have a fixed maximum volume and expulsion and intake of fluid may result from compressing and releasing pressure, respectively, on the outer walls of interior chamber 156.

In addition, the constraint or locking mechanisms associated with various aspects of the patency check devices 100 described herein, may be unidirectional or bidirectional. By unidirectional it is meant that the constraint or locking mechanism allows the patency check device 100 to do one of expand or contract, but not the other, when in the locked or constrained position. A bidirectional constraint or locking mechanism means a mechanism that controls or prevents both expansion and contraction when in the locked or constrained position. Most of the constraints shown in various aspects herein are unidirectional in that they resist expansion under the pressure of an injection but allow for contraction under the control of the operator, however, bidirectional constraint or locking mechanisms are envisioned. The forward most constraint of patency check device 100 illustrated in FIG. 5 is preferably a bidirectional constraint. With reference to FIG. 5 due to the design of the integral check valve 128, when the plunger rod 148 is pushed in, the first and second locking members 118 and 120 lock the piston rod 148 into a position with some volume in the chamber 112 to allow the valve 128 to open and prevent it from moving farther forward as well as backwards due to the pressure during injection. According to other aspects, the design of the proximal end 102 and the plunger 122 and slits 128 of the aspect of FIG. 5 may be designed and configured such that the patency check device can be fully compressed in the locked position and still allow the valve 128 to open. According to other aspects, for example that of FIGS. 10A-10D comprising a ratcheting mechanism that may be used to resist expansion but allow contraction all the way to the minimum volume possible.

According to some aspects, the patency check device 100 provides an inline expandable and shrinkable or contractible interior chamber, for example a piston in cooperation with a plunger or an expandable tube segment, that may be expanded to pull blood from a patient and or contractible to deliver fluid or blood to a patient. According to other aspects, fluid being delivered to the patient flows through the interior chamber 112 of the patency check device 100. According to some aspects, the fluid to be delivered is in fluid communication with the interior chamber 112 of the patency check device 100 during delivery. According to other aspects, the interior chamber 112 of the patency check device 100 is fluidly separate from the fluid delivery path during selected aspects of the fluid delivery process. If in fluid communication, the patency check device 100 locking mechanism and other aspects of the embodiments cooperate such that the expansion of the interior chamber 112 of the patency check device 100 is prevented or limited during fluid delivery, to ensure that most or all of the fluid is delivered to the patient.

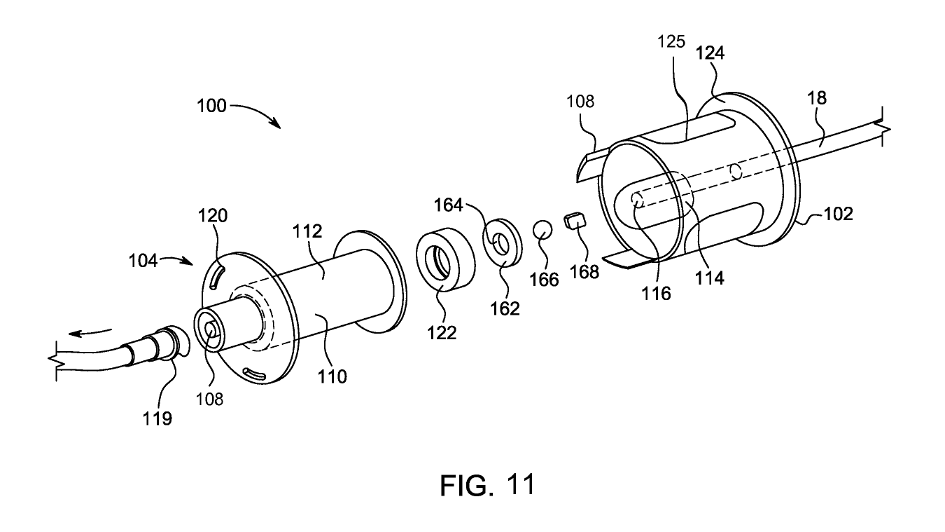

With reference to FIG. 11, a patency check device 100 is shown in accordance with a further aspect. The patency check device 100 has a proximal end 102 configured for connecting to the distal end 44 of the SPDS 18 (shown in FIG. 4A). The patency check device 100 further has a distal end 104 opposite to the proximal end 102. The distal end 104 of the patency check device 100 is configured for connecting to a needle 106 (shown in FIG. 4A) or to patient tubing 119 by way of a connector 108. The patency check device 100 has a tubular body 110 that defines an interior chamber 112 for drawing fluid during the patency check. A plunger tube 114 is slidably received within the interior chamber 112 of the tubular body 110. The plunger tube 114 has a hollow body with a fluid path 116 extending between the proximal and distal ends of the plunger tube 114.

With continuing reference to FIG. 11, the plunger tube 114 has an exterior casing 125 having a first locking member 118 provided on a distal surface thereof. The first locking member 118 is configured for interacting with a second locking member 120 provided at a distal end of the tubular body 110. In one aspect, first locking member 118 has a plurality of legs or tabs that extend circumferentially around the plunger tube 114 from the proximal end to the distal end. The second locking member 120 on the tubular body 110 is configured to operatively engage the plurality of legs of the first locking member 118. In one aspect, the second locking member 120 has a plurality of recesses or slots provided on a flange that extends circumferentially around the distal end 104 of the tubular body 110. Each of the recesses or slots is configured to receive and engage the corresponding leg or tab of the first locking mechanism 118 thereby placing the patency check device 100 in a locked configuration, for example by interaction of a projection on the leg or tab with the edge of the recess or slot. In certain aspects, once activated and locked, the connection between the first and second locking members 118, 120 may be non-reversible to prevent reuse of the patency check device 100.

With continuing reference to FIG. 11, the patency check device 100 further includes an annular seal 122 disposed around the distal end of the plunger tube 114. An annular valve seat 162 having a central opening 164 may be disposed within at least a portion of the annular seal 122. The annular valve seat 162 is configured to receive a valve 166, such as a ball valve, that is movable between a closed position, where the valve 166 prevents the passage of fluid through the central opening 164 of the annular valve seat 162, and an open position, where the valve 166 allows the passage of fluid through the central opening 164 of the annular valve seat 162. A spring 168 or other biasing element urges the valve 166 to the closed position. Once the patency check device 100 is placed in the locked position (i.e., locking of first and second locking members 118, 120), the valve 166 may be biased into the open position, for example by end stop 170, to allow fluid passage through central opening 164.

The proximal end of the plunger 114 has an activation member 124 that provides a pressing surface that can be engaged by one or more of the user's fingers. The plunger tube 114 of the patency check device 100 can be moved axially relative to the tubular body 110 to increase the volume of the interior chamber 112 and draw fluid (i.e., blood from the patients vasculature system, thereby confirming patency) into the interior chamber 112 (by moving the activation member 124 in a proximal direction) or to expel fluid from the interior chamber 112 (by moving the activation member 124 in a distal direction) and moving the patency check device 100 into the locked position.

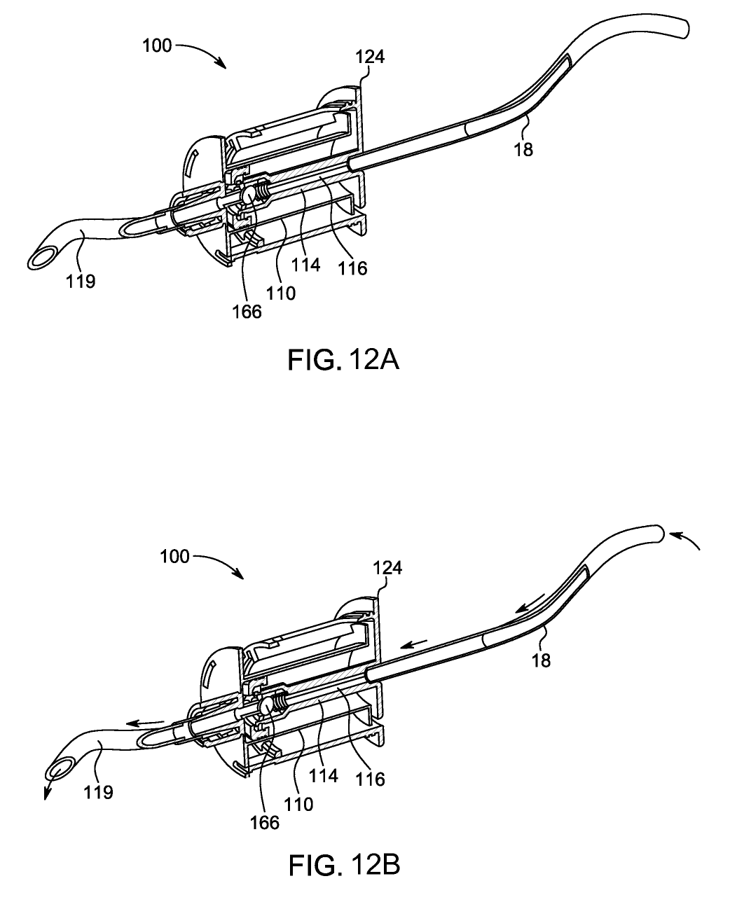

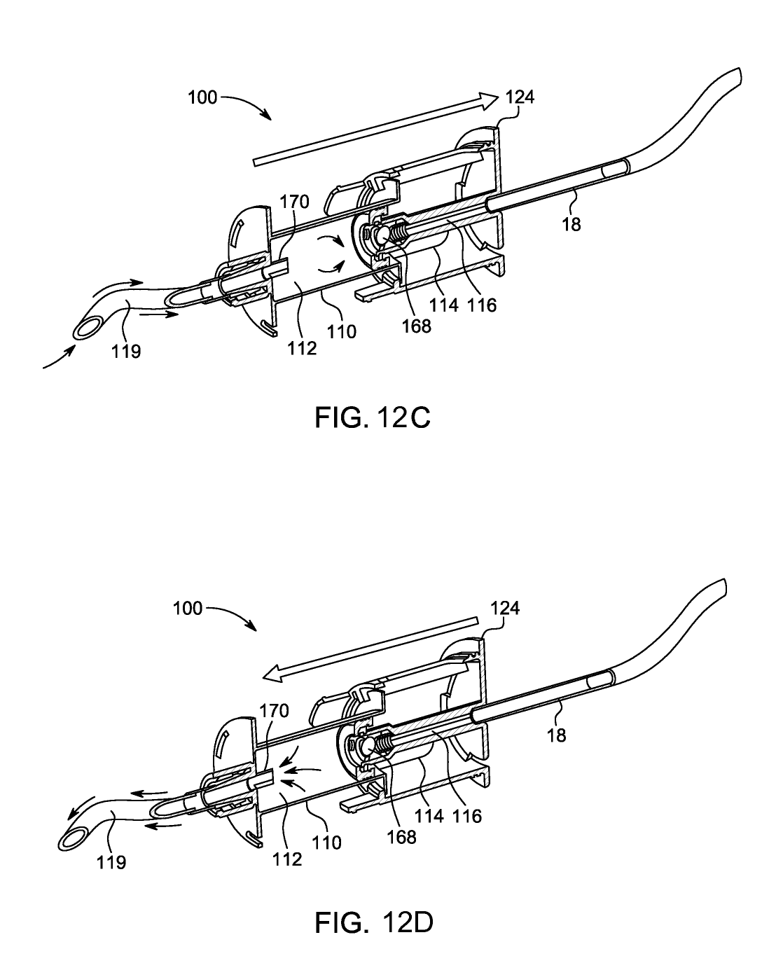

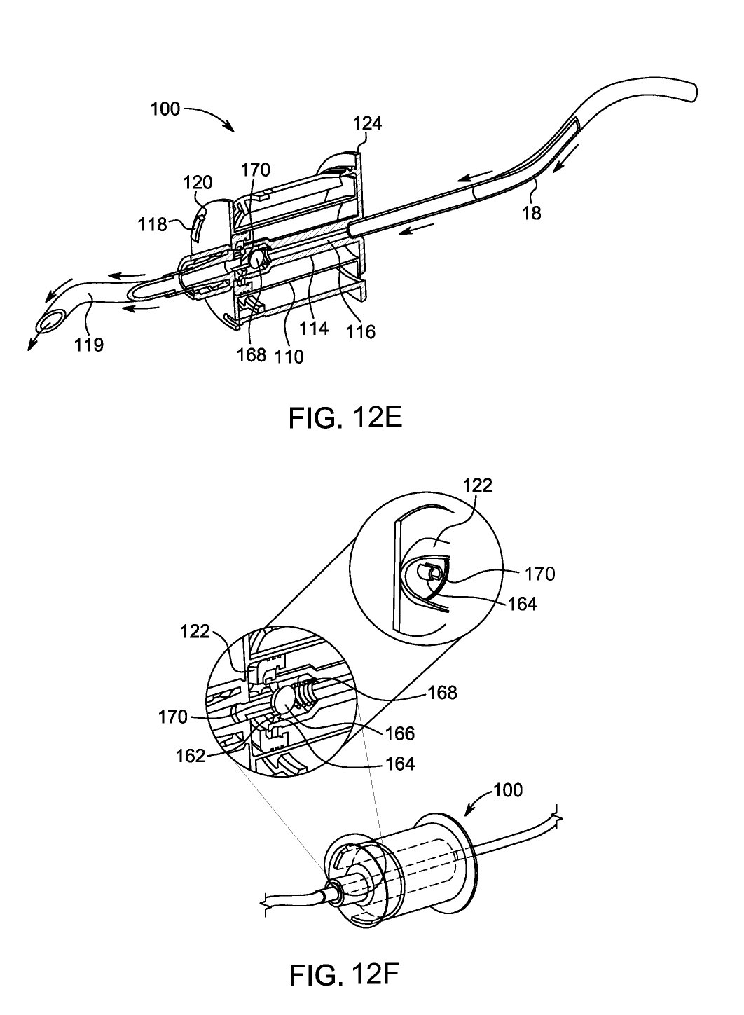

With reference to FIGS. 12A-12F, a patency check procedure will be described based on an aspect of the patency check device 100 shown in FIG. 11. Initially, with reference to FIG. 12A, the patency check device 100 is connected to the SPDS 18 and the patient tubing 119, and the valve 166 is in the open position (by end stop 170, see FIG. 12F) to allow the system to be primed. With reference to FIG. 12B, the SPDS 18 is primed by delivering fluid into the patency check device 100 such that the fluid fills the fluid path 116 and patient tubing 119. After inserting the needle or catheter 106 (shown in FIG. 4A) into the patient, the patency check procedure can be performed by urging the plunger 114 in a proximal direction relative to the body 110. Proximal movement of plunger 114 closes valve 166 and with the valve 166 in the closed position (FIG. 12C), fluid, such as blood from the patient's vasculature system, is drawn from the patient's body into the interior chamber 112 of the body 110, indicating whether patency has been achieved. If no blood is observed, then patency has not been achieved and the needle or catheter must be reinserted until patency is confirmed. After confirming the patency of the fluid connection with the patient's vasculature system, the plunger 114 is pushed in a distal direction such that fluid is expelled from the interior chamber 112 of the body 110 (FIG. 12D). The plunger tube 114 is then locked relative to the body 110 by connecting the first locking member 118 with the second locking member 120 (FIG. 12E). Upon full distal movement, the valve 166 is urged into an open position to allow the fluid to be delivered from the fluid delivery system 10 (shown in FIGS. 1-2) through SPDS 18 and patency check device 100 to the patient through patient tubing 119. With reference to FIG. 12F, according to certain aspects, the valve 166 may be urged into the open position by an end stop 170 on the distal end of the interior chamber 112. The end stop 170 urges the valve 166 away from the valve seat 162 to allow fluid to flow from SPDS 18 through the central opening 164 and into the interior chamber 112 and patient fluid path 119 before being delivered to the patient.

While various aspects of the inline patency check device were provided in the foregoing description, those skilled in the art may make modifications and alterations to these aspects without departing from the scope and spirit of the disclosure. For example, it is to be understood that this disclosure contemplates that, to the extent possible, one or more features of any aspect can be combined with one or more features of any other aspect. Accordingly, the foregoing description is intended to be illustrative rather than restrictive.

1. A fluid path set for use with an injection device, the fluid path set comprising:
a single-patient disposable set having a proximal end and a distal end, the proximal end configured for connecting with the injection device; and
a patency check device having a proximal end configured for connecting with the distal end of the single-patient disposable set and a distal end configured for connecting with a patient fluid delivery device, the patency check device comprising:
a body extending between the proximal end and the distal end of the patency check device, the body having a reversibly expandable interior chamber configured for drawing fluid through the patient fluid delivery device during a patency check; and
a locking mechanism configured for controlling the volume of the interior chamber during an injection procedure.

2. The fluid path set of claim 1, wherein the locking mechanism has a first locking member on a plunger slidably received within the body between the proximal end and the distal end of the patency check device and a second locking member on the body of the patency check device, wherein the locking mechanism controllably locks the plunger relative to the body of the patency check device.

3. The fluid path set of claim 1, wherein the proximal end of the patency check device has a first connector for releasably connecting with the single-patient disposable set.

4. The fluid path set of claim 1, wherein the distal end of the patency check device has a second connector for releasably connecting with the patient fluid delivery device.

5. The fluid path set of claim 1, wherein the proximal end of the patency check device is non-releasably connected with the single-patient disposable set.

6. The fluid path set of claim 1, wherein the distal end of the patency check device is non-releasably connected with the patient fluid delivery device.

7. The fluid path set of claim 2, wherein the plunger has a hollow body with a fluid path extending through the hollow body.

8. The fluid path set of claim 2, further comprising an annular seal between an outer portion of the plunger and an inner portion of the body of the patency check device.

9. The fluid path set of claim 8, further comprising a one-way check valve within the annular seal.

10. The fluid path set of claim 1, further comprising a removable cap at the distal end of the patency check device.

11. (canceled)

12. A patency check device comprising:
a proximal end configured for connecting with a distal end of a single-patient disposable set connectable to an injection device;
a distal end configured for connecting with a patient fluid delivery device;
a body extending between the proximal end and the distal end, the body having a reversibly expandable interior chamber configured for drawing fluid through the patient fluid delivery device during a patency check; and
a locking mechanism configured for controlling the volume of the interior chamber during an injection procedure.

13. The patency check device of claim 12, wherein the locking mechanism has a first locking member on a plunger slidably received within the body between the proximal end and the distal end of the patency check device and a second locking member on the body of the patency check device, wherein the locking mechanism controllably locks the plunger relative to the body of the patency check device.

14. The patency check device of claim 12, wherein the proximal end of the patency check device has a first connector for releasably connecting with the distal end of the single-patient disposable set.

15. The patency check device of claim 12, wherein the distal end of the patency check device has a second connector for releasably connecting with the patient fluid delivery device.

16. The patency check device of claim 12, wherein the plunger has a hollow body with a fluid path extending through the hollow body.

17. The patency check device of claim 13, further comprising an annular seal between an outer portion of the plunger and an inner portion of the body of the patency check device.

18. A method of determining patency of an injection site, the method comprising:
inserting a catheter or needle into a blood vessel of a patient;
connecting a patency check device into a fluid path between the catheter or needle and an injection device, the patency check device comprising:
a proximal end configured for connecting with a distal end of a single-patient disposable set connectable to the injection device;
a distal end configured for connecting with the catheter or needle or a patient fluid path connected to the catheter or needle;
a body extending between the proximal end and the distal end, the body having an interior chamber configured for drawing fluid from the blood vessel during a patency check;
a plunger slidably received within the body between the proximal end and the distal end; and
a locking mechanism configured for locking the plunger relative to the body to prevent movement of the plunger during an injection procedure;
withdrawing the plunger toward the proximal end of the body to check for patency, wherein patency is verified by drawing blood from the blood vessel into the interior chamber or into the patient fluid path; and
locking the plunger relative to the body to prevent movement of the plunger during an injection procedure.

19. The method of claim 18, further comprising priming the fluid path with a fluid from the injection device prior to withdrawing the plunger toward the proximal end of the body to check for patency.

20. The method of claim 18, further comprising injecting fluid from the injection device after locking the plunger relative to the body of the patency check device.

21. The method of claim 18, further comprising, if patency is present, backwashing blood from the interior chamber or the patient fluid path into the blood vessel of the patient with fluid from the injection device.
